(19) United States
(12) Patent Application Publication
(10) Pub. No.: US 2004/0064102 A1
(43) Pub. Date: Apr. 1, 2004

(54) Double-barrel syringe for ophthalmic surgeries

(76) Inventor: Akira Yamada, Fort Worth, TX (US)

Correspondence Address: Bausch & Lomb Incorporated, One Bausch & Lomb Place, Rochester, NY, 14604-2701, US

(21) Appl. No.: 10/262,491

(22) Filed: Sep. 30, 2002

(51) Int. Cl. A61M 5/00

(52) U.S. Cl. 604/191

(57) ABSTRACT

A syringe for use in ophthalmic surgery includes a pair of barrels and associated plungers which are easily and readily distinguishable by the surgeon allowing for selective and sequential delivery of two different material types through the same device.

### BACKGROUND OF THE INVENTION

The present invention relates to surgical instruments. More particularly, the present invention relates to a syringe having two barrels with associated plungers to permit selective, sequential delivery of two different materials loaded in the two barrels of the syringe. The inventive syringe is particularly suited for use delivering two materials which are typically used together in cataract eye surgery.

A common and desirable method of treating a cataract eye is to remove the clouded, natural lens and replace it with an artificial IOL in a surgical procedure known as cataract extraction. In the extracapsular extraction method, the natural lens is removed from the capsular bag while leaving the posterior part of the capsular bag (and preferably at least part of the anterior part of the capsular bag) in place within the eye. In this instance, the capsular bag remains anchored to the eye's ciliary body through the zonular fibers. In an alternate procedure known as intracapsular extraction, both the lens and capsular bag are removed in their entirety by severing the zonular fibers and replaced with an IOL which must be anchored within the eye absent the capsular bag. The intracapsular extraction method is considered less attractive as compared to the extracapsular extraction method since in the extracapsular method, the capsular bag remains attached to the eye's ciliary body and thus provides a natural centering and locating means for the IOL within the eye. The capsular bag also continues its function of providing a natural barrier between the aqueous humor at the front of the eye and the vitreous humor at the rear of the eye.

Many types of ophthalmic surgeries require use of various fluids which are both delivered and removed from the eye over the course of the surgery. For example, in cataract surgery, the surgeon will typically need to delivery a viscoelastic to the eye to protect the corneal endothelium and also to maintain the depth of the anterior chamber of the eye during the surgical procedure. Furthermore, different types and/or viscosities of viscoelastics may be used in the same surgery to serve different purposes. Examples of viscoelastic agents may be seen in the following patents:

U.S. Pat. No. 4,713,446 to DeVore et al on Dec. 15, 1987 entitled “Viscoelastic Collagen Solution For Ophthalmic Use And Method Of Preparation”;

U.S. Pat. No. 4,920,104 to DeVore et al on Apr. 24, 1990 entitled “Sodium Hyaluranate Composition”;

U.S. Pat. No. 5,166,331 to Valle et al on Nov. 24, 1992 entitled “Hyaluronics Acid Fractions, Methods For The Preparation Thereof, And Pharmaceutical Compositions Containing Same”; and

U.S. Pat. No. 5,631,243 to Kelman and DeVore on May 20, 1997 entitled “Collagen-Based Viscoelastic Solution For Viscoelastic Surgery”.

U.S. Pat. No. 5,792,103 to Schwartz et al on Aug. 11, 1998 entitled “Viscosurgical Method And Apparatus”, the entire disclosure of which is incorporated herein by reference, discloses a method and apparatus for administering a viscoelastic agent as well as a degradative agent during ophthalmic surgery. The viscoelastic is used in viscosurgery to protect the eye tissues and maintain chamber depth during the surgery. The viscoelastic may also be used to coat surgical instruments such as an IOL inserter to allow easy passage of an IOL through the inserter. When used in the eye, the viscoelastic usually causes an unwanted spike in intraocular pressure and the viscoelastic must be removed or degraded prior to closing the surgery in order to normalize pressure within the eye. In the '103 patent, this is done through the administration of a degradative agent which acts to remove the viscoelastic through various means, an example being enzymatic cleavage of a specific bond in a polymeric substrate. Specific examples of viscoelastic agents and associated degradative agents are listed in Table 2 of the '103 patent found at Column 6 thereof. It is further stated that the viscoelastic and degradative agent must be kept separate until they are injected to prevent loss of viscosity during the procedure. The '103 patent discloses an apparatus having a first chamber for holding the viscoelastic and a second chamber for holding the degradative agent which both lead to the same passage 18 where mixing occurs. Area 18 then leads to the tip for injection of the mixed materials into the eye. Separate shafts 28,29 connect to a common handle 26 for dispensing the materials from the two chambers through the tip. It thus appears that both materials are expressed at the same rate at the same time due to the common handle 26.

While the apparatus of the '103 patent may be useful in the particular applications given, it does not allow for the selective, sequential delivery of two different materials from the same device which is a desirable feature.

### SUMMARY OF THE INVENTION

The present invention provides an apparatus for the selective, sequential delivery of two materials from the same device into the eye or to coat a surgical apparatus. The apparatus comprises a syringe having first and second barrels positioned in side-by-side relationship including first and second plungers for telescoping within their respective chambers. The first and second plungers are independently movable through a thumb press provided at the proximal end of each plunger. The first and second chambers lead to a tip having a needle or cannula sized for inserting through the eye. To operate the device, the materials are loaded into their respective chambers and the surgeon selects and presses the plunger which is associated with the material the surgeon wishes to deliver to the eye or the surgical instrument. This delivers the selected material through the tip and into the eye or to the surgical instrument. When the surgeon wishes to delivery the second material into the eye or surgical instrument, the other plunger is pressed which delivers the second material through the tip and into the eye or surgical instrument. The plungers are provided in different lengths and/or diameters such that the different plungers and the materials they are associated with are easily and quickly distinguished by the surgeon, both through sight and through feel, during the surgery. The plungers may furthermore be color-coded to further enhance the visual distinguishing features of the device.

### BRIEF DESCRIPTION OF THE DRAWING

FIG. 1a is a side elevational view of a first embodiment of the inventive syringe;

FIG. 1b is the view of FIG. 1a with the syringe rotated 90°;

FIG. 1c is a cross-sectional view of FIG. 1a with the syringe rotated 180°;

FIG. 1d is a top plan view of the syringe taken in section through the barrels and plungers;

FIG. 2a is a side elevational view of a second embodiment of the inventive syringe;

FIG. 2b is the view of FIG. 2a with the syringe rotated 90°;

FIG. 2c is a cross-sectional view of FIG. 2a with the syringe rotated 180°; and

FIG. 2d is a top plan view of the syringe of FIG. 2a-c taken in section through the barrels and plungers.

### DETAILED DESCRIPTION

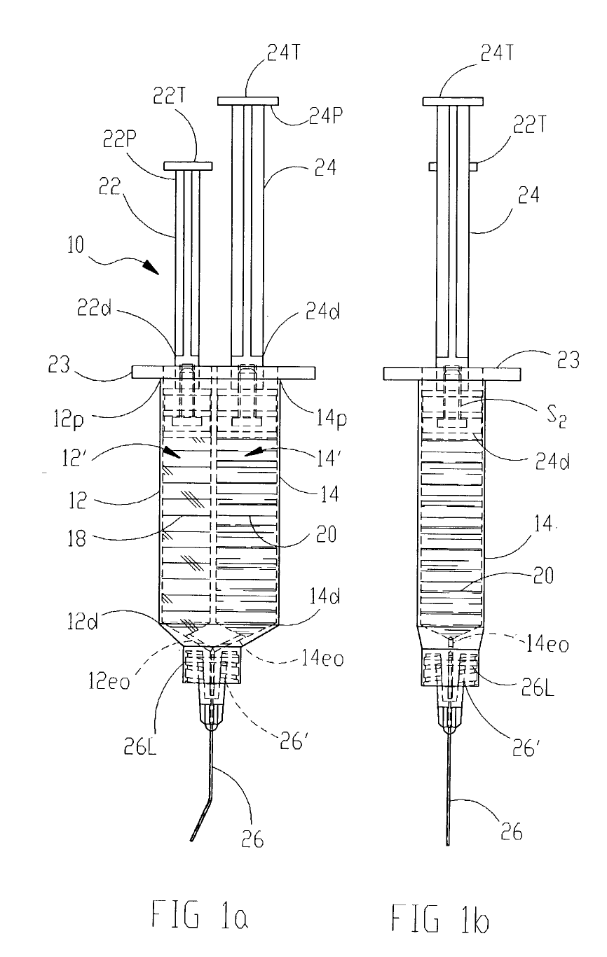

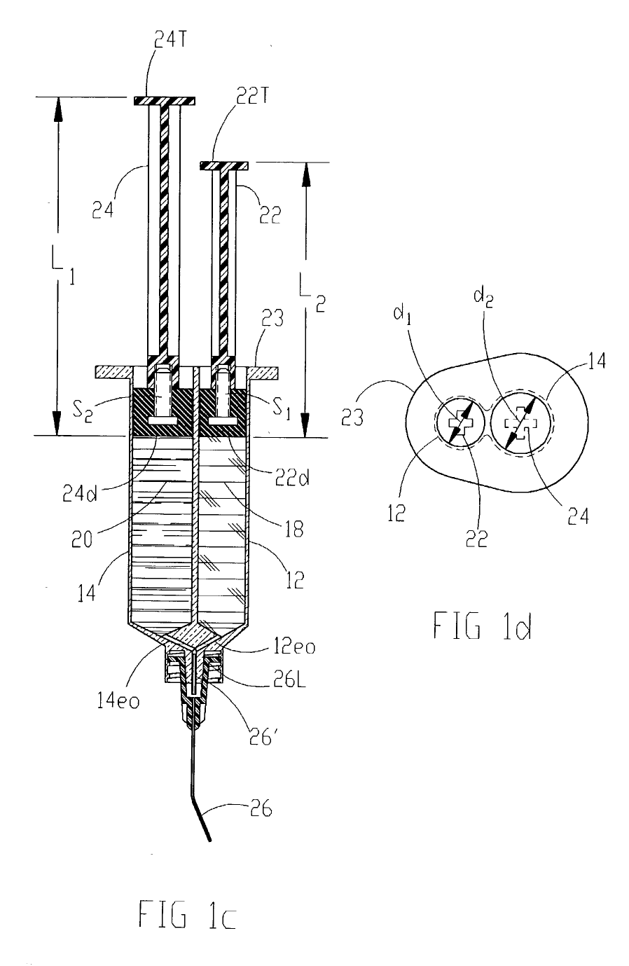

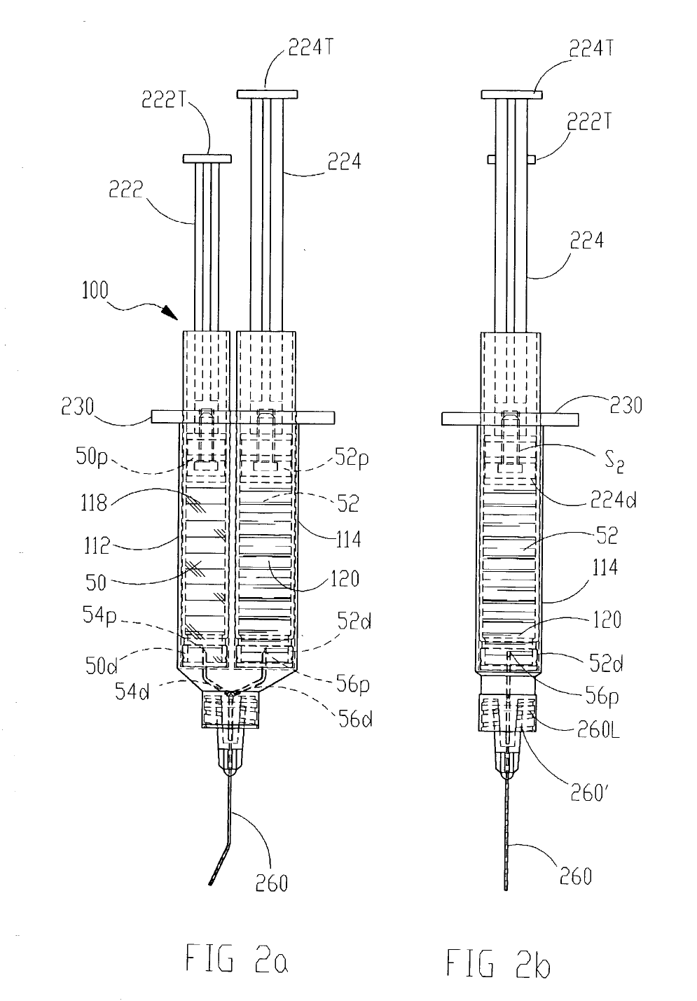

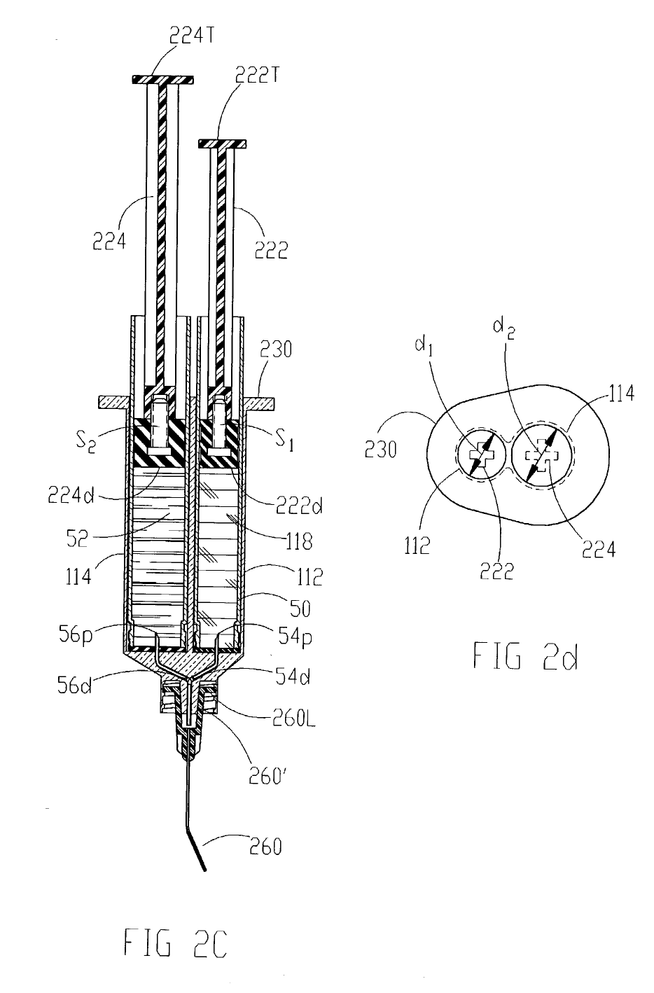

Referring now to the drawing, there is seen in FIGS. 1a-c and 2a-c first and second embodiments of the inventive syringe which differ in the respect that the first embodiment is configured for direct filling of the first and second materials inside their respective barrels while the second embodiment of FIG. 2a-c is configured for insertion of pre-filled first and second carpules into the first and second barrels, respectively. Like parts between the first and second embodiments are denoted by the same numerals multiplied by 10 for the second embodiment.

Referring to the first embodiment of FIG. 1a-c, the inventive syringe is denoted generally by the reference numeral 10. Syringe 10 is useful in ophthalmic surgery for selective, sequential delivery of first and second materials through the syringe and into the eye or to coat a surgical instrument. Examples of the two materials which may be delivered with syringe 10 include any type of liquefied materials which are typically used in the same surgical procedure. In the case of cataract surgery, examples of the first and second materials may be; (1) a viscoelastic and BSS (buffered saline solution); (2) sodium hyaluronate and sodium chondroitin sulfate; or (3) two viscoelastics having different viscoelastic characteristics. Any of the material combinations listed in Table 2 of the '103 patent may also be used with the present syringe.

Syringe 10 is seen to comprise first and second barrels 12, 14 each having an internal chamber 12′, 14′ for holding a quantity of first and second, liquefied materials 18, 20 therein, respectively. The first and second barrels 12, 14 are arranged in side-by-side relationship with each having opposite proximal 12p, 14p and distal 12d, 14d ends, respectively.

First and second plungers 22, 24 are provided with each having opposite proximal 22p, 24p and distal 22d, 24d ends, respectively. The first and second plungers 22, 24 are positioned for sliding engagement within the first and second barrels 12, 14 for telescoping movement therein, respectively. It is seen that the first and second plungers 22, 24 have different lengths L₁ and L₂ (FIG. 1c) to allow a surgeon using syringe 10 to easily distinguish between the first and second plungers 22, 24 when selecting which material 18 or 20 to eject through the syringe tip 26. The plungers may also be provided in different diameters D₁ and D₂ (FIG. 1d) as shown as an alternate to or additional to the different plunger lengths L₁ and L₂ for distinguishing between the two plungers 22, 24. The first and second plungers 22, 24 also each include a thumb press 22T, 24T attached to the proximal end thereof for manually operating the plungers by the surgeon. A flange 23 is provided at the proximal ends of the barrels 12p, 14p to assist in the manual manipulation of the syringe.

The syringe tip 26 is located adjacent the distal ends 12d, 14d of the first and second barrels 12, 14 and is in common, fluid communication therewith via exit orifices 12eo and 14eo. The exit orifices 12eo and 14eo provide the pathway for the first and second materials 18, 20, respectively, to the central orifice 26′ of tip 26 and thereby allowing passage of the first and second materials 18, 20 therethrough upon pressing the respective thumb press 22T, 24T of first and second plungers 22, 24 as selected by the surgeon. Tip 26 may be in the form of a needle directly attached to the syringe body, or may be in the form of a cannula which is attached to the syringe body via a Luer lock 26L.

Referring now to the second embodiment of the invention seen in FIG. 2a-d, the syringe is indicated generally by reference numeral 100. First and second carpules 50, 52 are provided and may be pre-filled with the first and second materials 180, 200 therein, respectively. The first and second carpules 50, 52 each include opposite proximal 50p, 52p and distal 50d, 52d ends and are removably insertable within said first and second barrels 112, 114, respectively. First and second syringe needles 54, 56 each having proximal 54p, 56p and distal 54d, 56d ends are located in the first and second barrels 112, 114 adjacent the distal ends 112d, 114d thereof, with the first and second syringe needle proximal ends 54p, 56p located within and extending toward said proximal ends of said first and second barrels 12p, 14p, respectively. As such, upon fully inserting the first and second carpules 50, 52 in the first and second barrels 112, 114, the proximal ends of said first and second syringe needles 54p, 56p pierce the carpule plug provided at the respective carpule distal end 50d, 52d and thereby accessing the material 118, 200 inside the carpule 50, 52, respectively. The distal ends of said first and second needles 54d, 56d extend into the tip 260 whereby the first and second materials 118, 200 may travel through a respective syringe needle 54, 56 and be expressed through the tip 260.

A novel syringe is thus provided which may be used for the selective, sequential delivery of first and second materials therethrough to an eye or surgical instrument. The materials chosen for use with the syringe will of course depend on the needs of the surgery being preformed.

What is claimed is:

1. A syringe for use in ophthalmic surgery for selective, sequential delivery of first and second materials through the syringe to an eye or surgical instrument, said syringe comprising:
a) first and second barrels each having an internal chamber for holding a quantity of said first and second materials therein, respectively, said first and second barrels lying in side-by-side relationship and each having opposite proximal and distal ends;
b) first and second plungers each having opposite proximal and distal ends, said first and second plungers positioned in said first and second barrels for telescoping movement therein, respectively, said first and second plungers having different lengths to allow a surgeon using said syringe to distinguish between the first and second plungers, said first and second plungers each having a thumb press attached to the proximal end thereof, respectively;
c) a tip located adjacent the distal ends of said barrels and in fluid communication therewith, said tip having a central orifice allowing passage of said first and second materials therethrough upon pressing the respective thumb press of said first and second plungers as selected by the surgeon.

2. The syringe of claim 1, and further comprising:
a) first and second carpules containing said first and second materials therein, respectively, said first and second carpules insertable within said first and second barrels, respectively;
b) first and second syringe needles each having proximal and distal ends and located in said first and second barrels adjacent said distal ends thereof with said first and second syringe needle proximal ends located within and extending toward said proximal ends of said first and second barrels, respectively, whereby insertion of said first and second carpules in said first and second barrels causes the proximal ends of said first and second syringe needles to pierce a respective said carpule thereby accessing the material inside the carpule, said distal ends of said first and second needles located within said tip whereby said first and second materials may travel through a respective said syringe needle and through said tip.

3. The syringe of claim 1 wherein said first and second plungers are of different colors.

4. The syringe of claim 1 wherein said first and second plungers and their associated barrels have different diameters.

5. The syringe of claim 1 wherein said first and second barrels are of different colors.

6. The syringe of claim 1 wherein said first material is a viscoelastic and said second material is BSS.

7. The syringe of claim 1 wherein said first material is sodium hyaluronate and said second material is sodium chondroitin sulfate.

8. The syringe of claim 1 wherein said first and second materials are viscoelastics having different viscoelastic characteristics.
